(19) United States
(12) Patent Application Publication
(10) Pub. No.: US 2019/0029507 A1
(43) Pub. Date: Jan. 31, 2019

(54) OPTICAL FIBER SCANNER, ILLUMINATION DEVICE, AND OBSERVATION DEVICE

(71) Applicant: OLYMPUS CORPORATION, Tokyo (JP)

(72) Inventors: Hirokazu YOKOTA, Tokyo (JP); Yasuaki KASAI, Saitama (JP); Hiroshi TSURUTA, Kanagawa (JP); Takashi YASUMI, Tokyo (JP)

(73) Assignee: OLYMPUS CORPORATION, Tokyo (JP)

(21) Appl. No.: 16/139,145

(22) Filed: Sep. 24, 2018

Related U.S. Application Data
(63) Continuation of application No. PCT/JP2016/082309, filed on Oct. 31, 2016.

(30) Foreign Application Priority Data
Mar. 31, 2016 (JP) PCT/JP2016/060813

(51) Int. Cl. A61B 1/06; G02B 23/26; G02B 26/10; H01L 41/09; A61B 1/00; A61B 1/07

(52) U.S. Cl. CPC A61B 1/0615; G02B 23/26; A61B 1/0676; A61B 5/0062; H01L 41/09; A61B 1/00188; A61B 1/07; G02B 26/103

(57) ABSTRACT

An optical fiber scanner includes an optical fiber and a fixed member that is fixed to the outer periphery of the optical fiber. The fixed member is provided with: two vibration parts that are adjacent to the optical fiber in radial directions of the optical fiber that intersect with each other and that expand and contract in the longitudinal direction of the optical fiber; and a connector that connects the two vibration parts. The two vibration parts respectively have inner surfaces that abut against the outer periphery of the optical fiber.

### CROSS-REFERENCE TO RELATED APPLICATIONS

This is a continuation of International Application PCT/JP2016/082309, with an international filing date of Oct. 31, 2016, which is hereby incorporated by reference herein in its entirety.

### TECHNICAL FIELD

The present invention relates to an optical fiber scanner, an illumination device, and an observation device.

### BACKGROUND ART

In the related art, there is a known optical fiber scanner that is provided with an optical fiber and piezoelectric elements fixed to the outer periphery of the optical fiber and in which the optical fiber is made to undergo a bending vibration through expansion and contraction of the piezoelectric elements, thus scanning light emitted from the distal end of the optical fiber (for example, see PTL 1). In order to two-dimensionally scan the light, it is necessary to produce a bending vibration in the optical fiber in two axial directions perpendicular to each other (X-direction and Y-direction). Thus, in the optical fiber scanner of PTL 1, the X-direction piezoelectric elements and the Y-direction piezoelectric elements are disposed at positions shifted by 90 degrees in the circumferential direction of the optical fiber.

### CITATION LIST

### Patent Literature

{PTL 1} Japanese Unexamined Patent Application, Publication No. 2014-71423

### SUMMARY OF INVENTION

### Technical Problem

An object of the present invention is to provide an optical fiber scanner capable of obtaining stable scanning performance by improving assembly accuracy, and an illumination device and an observation device that are provided with the same.

### Solution to Problem

According to a first aspect, the present invention provides an optical fiber scanner including: an optical fiber that has a longitudinal axis and that emits light from a distal end portion thereof; and a fixed member that is fixed to an outer periphery of the optical fiber at a position closer to a base end portion of the optical fiber than to the distal end portion thereof, wherein the fixed member is provided with: a first vibration part that is adjacent to the optical fiber in a first radial direction of the optical fiber and that expands and contracts in the longitudinal direction of the optical fiber when a voltage is applied thereto; a second vibration part that is adjacent to the optical fiber in a second radial direction of the optical fiber, the second radial direction intersecting with the first radial direction, and that expands and contracts in the longitudinal direction of the optical fiber when a voltage is applied thereto; and a connector that connects the first vibration part and the second vibration part; the first vibration part has a first inner surface that abuts against the outer periphery of the optical fiber; and the second vibration part has a second inner surface that is disposed at an angle with respect to the first inner surface and that abuts against the outer periphery of the optical fiber.

In the above-described first aspect, at least one part of the connector may have a higher vibration absorption than the vibration absorption of the first vibration part and the second vibration part.

In the above-described first aspect, it is preferable that an outer part of the connector that is located on the opposite side from the optical fiber have a higher vibration absorption than the vibration absorption of the first vibration part and the second vibration part.

In the above-described first aspect, the fixed member may have a third inner surface that is provided at the opposite side of the optical fiber from the first inner surface and that is brought into contact with the outer periphery of the optical fiber.

The above-described first aspect may further include a third vibration part that is provided at the opposite side of the optical fiber from the first vibration part and that expands and contracts in the longitudinal direction of the optical fiber when a voltage is applied thereto, wherein the third vibration part may have the third inner surface.

In the above-described first aspect, the fixed member may be open, in the second radial direction, at the opposite side of the optical fiber from the second inner surface.

In the above-described first aspect, the first inner surface and the third inner surface may have a larger size than the radius of the optical fiber in the second radial direction; and an opening of the fixed member on the opposite side of the optical fiber from the second inner surface may have a larger size than the diameter of the optical fiber in the direction perpendicular to the second radial direction.

In the above-described first aspect, the size of the second vibration part in the second radial direction may be larger than the sizes of the first vibration part and the third vibration part in the first radial direction.

In the above-described first aspect, the fixed member may include: a first fixed member that is provided with the first vibration part, the second vibration part, and the connector; and a second fixed member that is provided with a fourth vibration part having a fourth inner surface that is provided at the opposite side of the optical fiber from the second inner surface and that is brought into contact with the outer periphery of the optical fiber; and the first fixed member and the second fixed member may be disposed adjacent to each other in the circumferential direction around the longitudinal axis and may be assembled into a tube shape surrounding the outer periphery of the optical fiber, in a close contact state.

In the above-described first aspect, the fixed member may include: a first fixed member that is provided with the first vibration part, the second vibration part, and the connector; and a second fixed member that is provided with: a third vibration part having a third inner surface that is provided at the opposite side of the optical fiber from the first inner surface and that is brought into contact with the outer periphery of the optical fiber; a fourth vibration part having a fourth inner surface that is provided at the opposite side of the optical fiber from the second inner surface and that is brought into contact with the outer periphery of the optical fiber; and a second connector that connects the third vibration part and the fourth vibration part; and the first fixed member and the second fixed member may be adjacent to each other in the circumferential direction around the longitudinal axis and may be assembled into a tube shape surrounding the outer periphery of the optical fiber, in a close contact state.

In the above-described first aspect, the first fixed member and the second fixed member may have, at both ends thereof in the circumferential direction, chamfered parts that at least partially extend along the longitudinal direction; and the first fixed member and the second fixed member may be assembled in a close contact state such that the chamfered parts are brought into close contact with each other.

In the above-described first aspect, a metal coating for coating the outer periphery of the optical fiber from the base end of the optical fiber to a section thereof in the vicinity of the distal end of the fixed member may be provided on the outer periphery of the optical fiber.

In the above-described first aspect, the optical fiber may have a square-prism shape having side surfaces in the first radial direction and the second radial direction.

According to a second aspect, the present invention provides an illumination device including: a light source that produces illumination light; and an optical fiber scanner according to the first aspect, in which the base end of the optical fiber is connected to the light source.

According to a third aspect, the present invention provides an observation device including: an illumination device according to the second aspect; a light detector that detects return light returning from an object when the object is irradiated with illumination light from the illumination device; and a voltage supplyer that supplies the voltage to the first vibration part and the second vibration part of the fixed member.

### BRIEF DESCRIPTION OF DRAWINGS

FIG. 1 is a view showing the overall configuration of an observation device according to a first embodiment of the present invention.

FIG. 2 is a longitudinal sectional view showing an internal structure of the distal end of an insertion portion of an endoscope in the observation device shown in FIG. 1, along the longitudinal axis thereof.

FIG. 3 is a front view of an optical fiber scanner according to the first embodiment of the present invention, viewed from the distal end thereof in the longitudinal-axis direction.

FIG. 4 is a front view of a fixed member shown in FIG. 3, viewed from the distal end thereof in the longitudinal-axis direction.

FIG. 5 is a front view of a modification of the fixed member shown in FIG. 3, viewed from the distal end thereof in the longitudinal-axis direction.

FIG. 6 is a front view of another modification of the fixed member shown in FIG. 3, viewed from the distal end thereof in the longitudinal-axis direction.

FIG. 7 is a front view of still another modification of the fixed member shown in FIG. 3, viewed from the distal end thereof in the longitudinal-axis direction.

FIG. 8 is a front view of still another modification of the fixed member shown in FIG. 3, viewed from the distal end thereof in the longitudinal-axis direction.

FIG. 9 is a front view of an optical fiber scanner according to a second embodiment of the present invention, viewed from the distal end thereof in the longitudinal-axis direction.

FIG. 10 is a front view of a fixed member shown in FIG. 9, viewed from the distal end thereof in the longitudinal-axis direction.

FIG. 11 is a front view of a modification of a fixed member shown in FIG. 10, viewed from the distal end thereof in the longitudinal-axis direction.

FIG. 12 is a front view of another modification of the fixed member shown in FIG. 10, viewed from the distal end thereof in the longitudinal-axis direction.

FIG. 13 is an exploded front view of a modification of a fixed member shown in FIG. 8, viewed from the distal end thereof in the longitudinal-axis direction.

### DESCRIPTION OF EMBODIMENTS

### First Embodiment

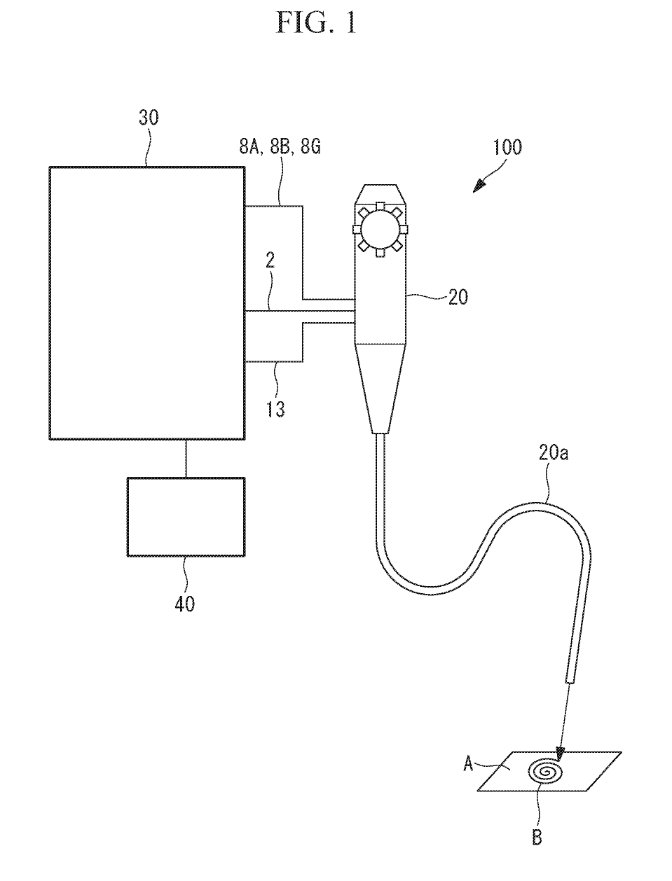

An optical fiber scanner 1, an illumination device 10, and an observation device 100 according to a first embodiment of the present invention will be described below with reference to FIGS. 1 to 8.

As shown in FIG. 1, the observation device 100 of this embodiment is provided with: an endoscope 20 that has an elongated insertion portion 20a; a control device body 30 that is connected to the endoscope 20; and a display 40 that is connected to the control device body 30. The observation device 100 is an optical-scanning-type endoscope device that scans illumination light emitted from the distal end of the insertion portion 20a of the endoscope 20, on an object A along a spiral scanning trajectory B, and that acquires an image of the object A.

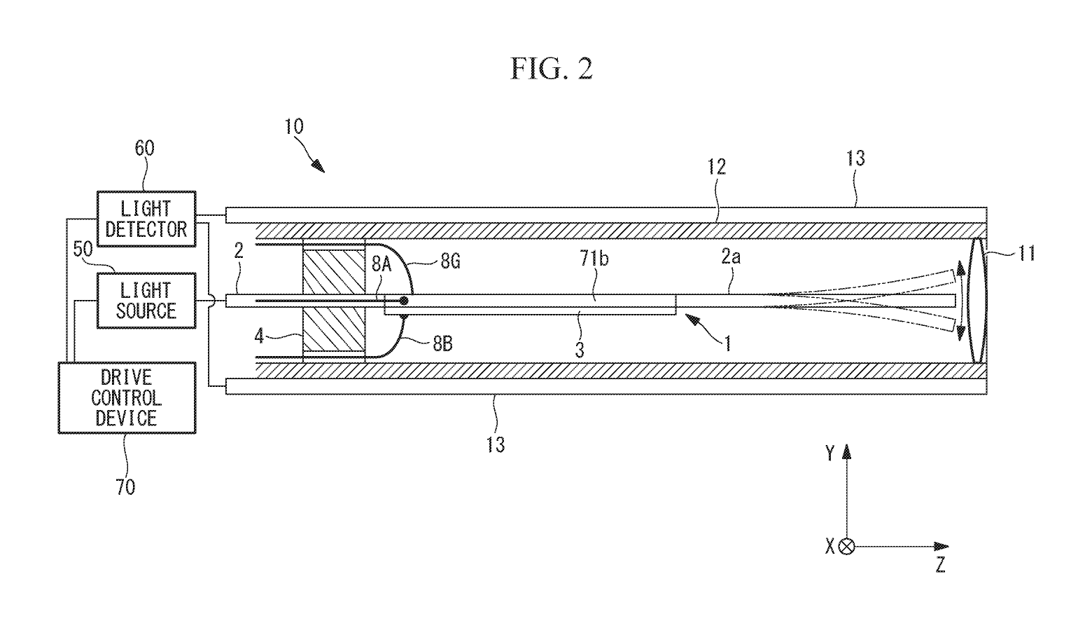

As shown in FIG. 2, the observation device 100 is further provided with: the illumination device 10, which radiates illumination light onto the object A; a light detector 60, such as a photodiode, that detects return light returning from the object A when the object A is irradiated with the illumination light; and a drive control device (voltage supplyer) 70 that drivingly controls the illumination device 10 and the light detector 60. The light detector 60 and the drive control device 70 are provided in the control device body 30.

The illumination device 10 is provided with: a light source 50 that is provided in the control device body 30 and that produces illumination light; the optical fiber scanner 1, which is provided in the insertion portion 20a and which has an illumination optical fiber 2 that guides the illumination light produced by the light source 50 and that emits the illumination light from the distal end thereof; a focusing lens 11 that is disposed closer to the distal end than the optical fiber 2 is and that focuses the illumination light emitted from the optical fiber 2; an elongated tubular frame 12 that accommodates the optical fiber scanner 1 and the focusing lens 11; and a plurality of detection optical fibers 13 that are provided on the outer periphery of the frame 12 in an arranged manner in the circumferential direction and that guide return light (for example, reflected light of the illumination light or fluorescence) from the object A to the light detector 60.

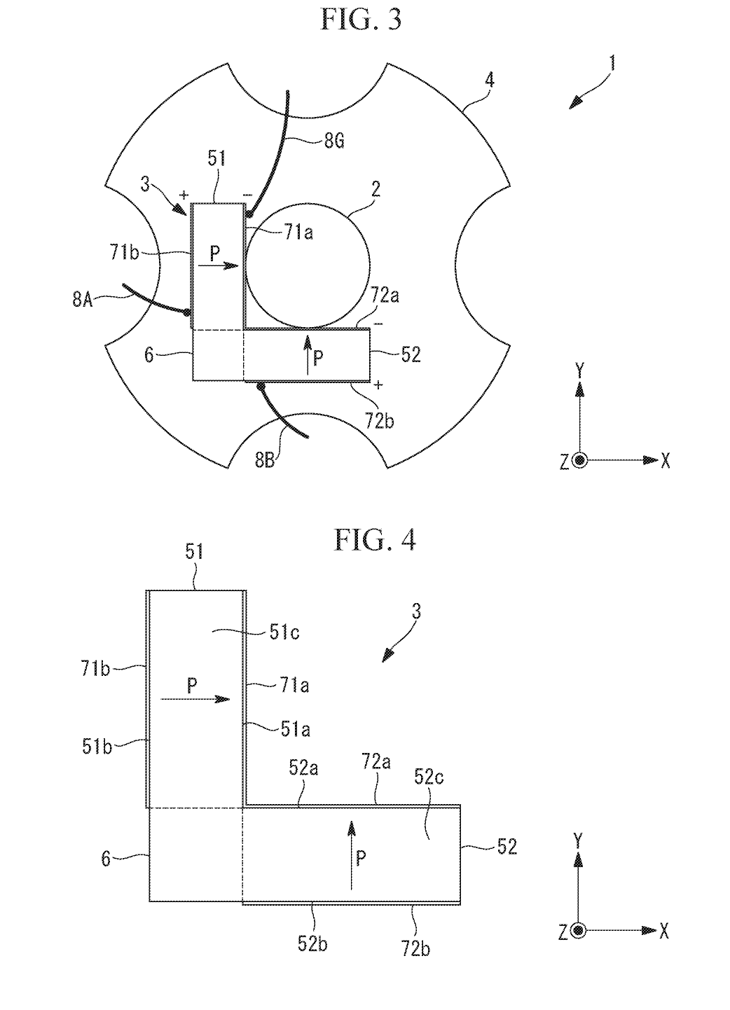

As shown in FIGS. 2 and 3, the optical fiber scanner 1 is provided with: the optical fiber 2; a fixed member 3 that is fixed to the outer periphery of the optical fiber 2; and a fixing part 4 that is provided on the optical fiber 2 at a position closer to the base end than the fixed member 3 is and that fixes the optical fiber 2 to the frame 12.

The optical fiber 2 is a multi-mode fiber or a single-mode fiber and is made of a cylindrical glass material having a longitudinal axis. The optical fiber 2 is disposed along the longitudinal direction of the frame 12 and extends from the base end of the frame 12 to the control device body 30. The distal end of the optical fiber 2 is disposed, inside the frame 12, in the vicinity of a distal end portion, and the base end of the optical fiber 2 is connected to the light source 50 in the control device body 30.

The fixed member 3 is provided on the outer periphery of the optical fiber 2 at a position closer to the base end of the optical fiber 2 than to the distal end of the optical fiber 2, and a distal end section (hereinafter, referred to as “protrusion”) 2a of the optical fiber 2 protrudes from a distal-end surface of the fixed member 3. Hereinafter, the longitudinal direction of the optical fiber 2 is referred to as the Z-direction, and two radial directions of the optical fiber 2 that are perpendicular to each other are referred to as the X-direction (first radial direction) and the Y-direction (second radial direction).

As shown in FIGS. 3 and 4, the cross-sectional shape of the fixed member 3 in an XY-plane is an L-shape in which two flat inner surfaces 51a and 52a that are perpendicular to each other are brought into contact with the cylindrical outer periphery of the optical fiber 2. The two inner surfaces 51a and 52a and the outer periphery of the optical fiber 2 are fixed with an adhesive agent. The fixed member 3 is made of an entirely-homogeneous piezoelectric material (for example, lead zirconate titanate) and has a seamless integral structure. The fixed member 3 is manufactured by being cut out from a prismatic piezoelectric material, for example.

The fixed member 3 is composed of: a first vibration part 51 that is adjacent to the optical fiber 2 in the X-direction; a second vibration part 52 that is adjacent to the optical fiber 2 in the Y-direction; and a connector 6 that connects the first vibration part 51 and the second vibration part 52. The connector 6 is provided between an end of the first vibration part 51 that is located close to the second vibration part 52 and an end of the second vibration part 52 that is located close to the first vibration part 51.

The distal end and the base end of the fixed member 3 are open in the Z-direction. The fixed member 3 is open at the opposite sides of the optical fiber 2 from the first vibration part 51 and the second vibration part 52 in the X-direction and the Y-direction, respectively. Therefore, in the assembly process of the optical fiber 2 and the fixed member 3, the optical fiber 2 is brought toward the inner surfaces 51a and 52a in a radial direction, thus being made to abut against the inner surfaces 51a and 52a.

The first vibration part 51 has: the flat first inner surface 51a, against which the outer periphery of the optical fiber 2 is made to abut; and a first outer surface 51b that is located closer to an outer side than the first inner surface 51a is in the X-direction and that is opposed to the first inner surface 51a. The first inner surface 51a is disposed parallel to the Z-direction, along a tangent to the outer periphery of the optical fiber 2 in the Y-direction. Electrodes 71a and 71b are formed on the first inner surface 51a and the first outer surface 51b, respectively, and the piezoelectric material is polarized in the X-direction in a region between the first inner surface 51a and the first outer surface 51b. Accordingly, a first piezoelectrically active region 51c that expands and contracts in the Z-direction through application of a voltage between the electrodes 71a and 71b is formed between the first inner surface 51a and the first outer surface 51b. Arrows P in the figure indicate polarization directions of the piezoelectric material.

The second vibration part 52 has: the flat second inner surface 52a, against which the outer periphery of the optical fiber 2 is made to abut; and a second outer surface 52b that is located closer to an outer side than the second inner surface 52a is in the Y-direction and that is opposed to the second inner surface 52a. The second inner surface 52a is disposed parallel to the Z-direction, along a tangent to the outer periphery of the optical fiber 2 in the X-direction. Electrodes 72a and 72b are formed on the second inner surface 52a and the second outer surface 52b, respectively, and the piezoelectric material is polarized in the Y-direction in a region between the second inner surface 52a and the second outer surface 52b. Accordingly, a second piezoelectrically active region 52c that expands and contracts in the Z-direction through application of a voltage between the electrodes 72a and 72b is formed between the second inner surface 52a and the second outer surface 52b.

The electrodes 71a and 71b of the first vibration part 51 are each disposed across the central axis of the optical fiber 2 in the Y-direction. It is preferable that the electrodes 71a and 71b each be disposed such that the sizes thereof on both sides of the central axis of the optical fiber 2 become equal.

The electrodes 72a and 72b of the second vibration part 52 are each disposed across the central axis of the optical fiber 2 in the X-direction. It is preferable that the electrodes 72a and 72b each be disposed such that the sizes thereof on both sides of the central axis of the optical fiber 2 become equal.

A phase-A lead 8A is connected to the electrode 71b, a phase-B lead 8B is connected to the electrode 72b, and a GND lead 8G is connected to the electrode 71a or the electrode 72a. The leads 8A, 8B, and 8G are connected to the drive control device 70 in the control device body 30. A conductive adhesive agent or solder is used for the connections between the electrodes 71a, 71b, 72a, and 72b and the leads 8A, 8B, and 8G.

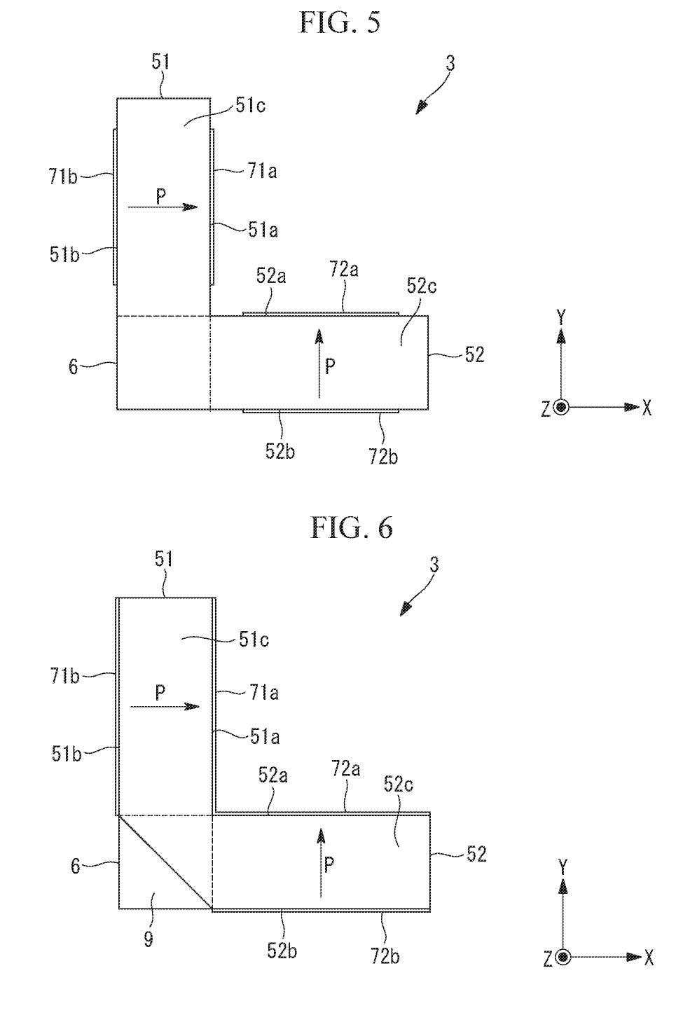

Although the electrodes 71a and 72a, shown in FIGS. 3 and 4, are formed on the entire inner surfaces 51a and 52a, respectively, and are connected to each other, instead of this, as shown in FIG. 5, the electrodes 71a and 72a may be formed only on partial sections of the inner surfaces 51a and 52a, respectively, and may be separated from each other. In this case, GND leads 8G are each connected to the electrode 71a and the electrode 72a. The electrodes 71b and 72b, which are formed on the outer surfaces 51b and 52b, may also be formed only on partial sections of the outer surfaces 51b and 52b, as shown in FIG. 5.

The fixing part 4 is formed of a cylindrical member and is provided on the radially outer side of the optical fiber 2. The inner periphery of the fixing part 4 is fixed to the outer periphery of the optical fiber 2 by means of an adhesive agent, and the outer periphery of the fixing part 4 is fixed to an inner wall of the frame 12. Accordingly, the optical fiber 2 is supported by the fixing part 4 in a cantilevered manner, in which the distal end thereof is a free end.

The drive control device 70 applies a phase-A alternating voltage having a predetermined driving frequency to the electrode 71b via the phase-A lead 8A and applies a phase-B alternating voltage having the predetermined driving frequency to the electrode 72b via the phase-B lead 8B. The predetermined driving frequency is set to the same frequency as the natural frequency of the protrusion 2a of the optical fiber 2 or to a frequency close to the natural frequency. Here, the drive control device 70 supplies the phase-A alternating voltage and the phase-B alternating voltage, whose phases differ from each other by π/2 and whose amplitudes temporarily vary in a sinusoidal manner, to the phase-A lead 8A and the phase-B lead 8B, respectively.

Next, the operation of the thus-configured optical fiber scanner 1, the illumination device 10, and the observation device 100 will be described.

In order to observe the object A by using the observation device 100 of this embodiment, the drive control device 70 is actuated to cause illumination light to be supplied from the light source 50 to the optical fiber 2 and to cause the alternating voltages, having the predetermined driving frequency, to be applied to the electrodes 71b and 72b via the leads 8A and 8B.

When the phase-A alternating voltage is supplied to the electrode 71b, and the voltage is applied to the first piezoelectrically active region 51c in the X-direction, the first piezoelectrically active region 51c undergoes a stretching vibration in the Z-direction perpendicular to the polarization direction, thus exciting, at the protrusion 2a of the optical fiber 2, an X-direction bending vibration in which the position of the fixing part 4 serves as a node, and the distal end of the optical fiber 2 serves as an antinode, and thus making the distal end of the optical fiber 2 vibrate in the X-direction. Accordingly, illumination light emitted from the distal end of the optical fiber 2 is linearly scanned in the X-direction.

When the phase-B alternating voltage is supplied to the electrode 72b, and the alternating voltage is applied to the second piezoelectrically active region 52c in the Y-direction, the second piezoelectrically active region 52c undergoes a stretching vibration in the Z-direction perpendicular to the polarization direction, thus exciting, at the protrusion 2a of the optical fiber 2, a Y-direction bending vibration in which the position of the fixing part 4 serves as a node, and the distal end of the optical fiber 2 serves as an antinode, and thus making the distal end of the optical fiber 2 vibrate in the Y-direction. Accordingly, illumination light emitted from the distal end of the optical fiber 2 is linearly scanned in the Y-direction.

Here, the phase of the phase-A alternating voltage and the phase of the phase-B alternating voltage are shifted from each other by π/2, and the amplitudes of the phase-A alternating voltage and the phase-B alternating voltage temporarily vary in a sinusoidal manner, thereby causing the distal end of the optical fiber 2 to vibrate along a spiral trajectory and two-dimensionally scanning illumination light on the object A along the spiral trajectory. Because the driving frequency is equivalent to the natural frequency of the protrusion 2a or is a frequency close thereto, the protrusion 2a can be efficiently excited.

Return light from the object A is received by the plurality of optical fibers 13, and the intensity thereof is detected by the light detector 60. The drive control device 70 causes the light detector 60 to detect the return light in synchronization with the scanning period of the illumination light and associates the intensity of the detected return light with the scanning position of the illumination light, thereby generating an image of the object A. The generated image is output from the control device body 30 to the display 40 and is displayed on the display 40.

Here, an assembly method for the optical fiber scanner 1 will be described.

In order to assemble the optical fiber scanner 1 of this embodiment, an adhesive agent is applied to the first inner surface 51a and the second inner surface 52a of the fixed member 3. Next, the relative positions of the optical fiber 2 and the fixed member 3 are set so as to make the outer periphery of the optical fiber 2 abut against both the first inner surface 51a and the second inner surface 52a. Next, the adhesive agent is hardened, thus fixing the fixed member 3 to the outer periphery of the optical fiber 2. Accordingly, the optical fiber 2 and the fixed member 3 can be assembled.

In this case, according to this embodiment, the two vibration parts 51 and 52, which cause the optical fiber 2 to undergo a bending vibration in the X-direction and the Y-direction, are sections of the fixed member 3, which is formed of a single member. Specifically, the relative positions of the first vibration part 51 and the second vibration part 52 are fixed. The outer periphery of the optical fiber 2 is made to abut against the two inner surfaces 51a and 52a of the fixed member 3, which are perpendicular to each other, thereby positioning the optical fiber 2 at a predetermined position with respect to the fixed member 3. Therefore, the relative positions of the first vibration part 51, the second vibration part 52, and the optical fiber 2 can be uniquely set. Accordingly, there is an advantage in that it is possible to improve the assembly accuracy of the optical fiber scanner 1 and to stably manufacture the optical fiber scanner 1 having a desired scanning performance.

In this embodiment, although the fixed member 3 is formed of a seamless integral structure, instead of this, it is also possible to join three blocks that constitute the first vibration part 51, the second vibration part 52, and the connector 6, thereby forming a fixed member 3 that is formed of a single member as a whole.

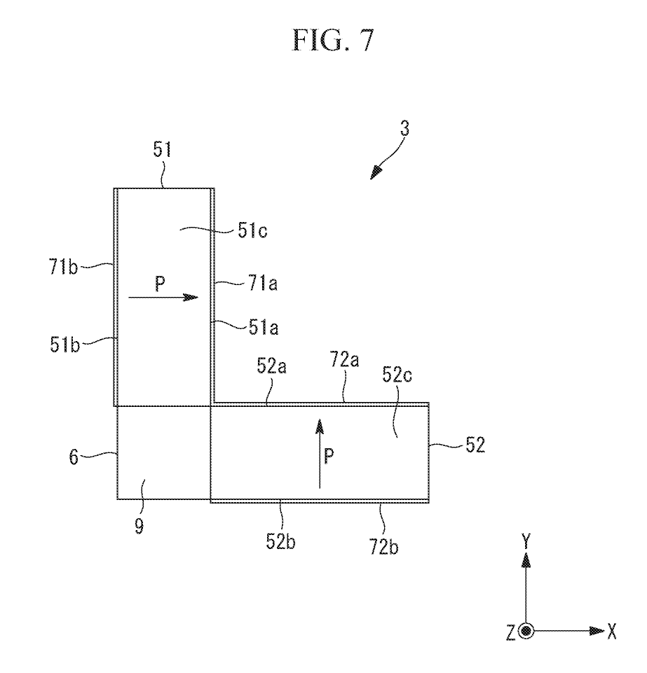

In this embodiment, although the whole connector 6 is made of a piezoelectric material, instead of this, as shown in FIGS. 6 and 7, at least a part of the connector 6 may be constituted of a vibration absorbing member 9 that has a higher vibration absorption than at least one of the first vibration part 51 and the second vibration part 52. In the example shown in FIG. 6, only a part of the connector 6 is constituted of the vibration absorbing member 9. In the example shown in FIG. 7, the whole connector 6 is constituted of the vibration absorbing member 9.

By doing so, it is possible to suppress transmission of vibrations between the first piezoelectrically active region 51c and the second piezoelectrically active region 52c. It is preferable that the material of the vibration absorbing member 9 be hard resin, for example, PEEK, engineering plastic, or elastomer.

As shown in FIG. 6, when the vibration absorbing member 9 is provided only partially on the connector 6, it is preferable that the vibration absorbing member 9 be provided on an outer part of the connector 6 that is located on the opposite side from the optical fiber 2. By doing so, it is possible to facilitate machining of the connector 6.

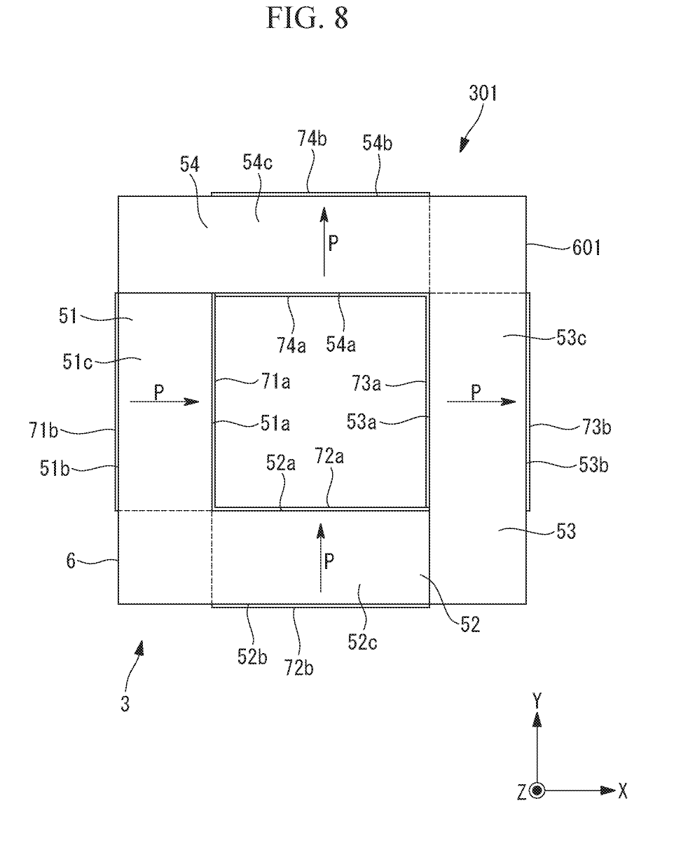

In this embodiment, although the single fixed member 3 is provided, instead of this, as shown in FIG. 8, it is also possible to further provide another fixed member (second fixed member) 301 that is disposed adjacent to the fixed member (first fixed member) 3 in the circumferential direction so as to form a square tube surrounding the optical fiber 2 together with the fixed member 3.

As in the first fixed member 3, the second fixed member 301 has, in an XY-plane, an L-shape in cross-section in which two flat inner surfaces 53a and 54a that are perpendicular to each other are brought into contact with the outer periphery of the optical fiber 2.

The second fixed member 301 is composed of: a third vibration part 53 that is adjacent to the optical fiber 2 in the X-direction at the opposite side from the first vibration part 51; a fourth vibration part 54 that is adjacent to the optical fiber 2 in the Y-direction at the opposite side from the second vibration part 52; and a connector (second connector) 601 that connects the third vibration part 53 and the fourth vibration part 54.

The third vibration part 53 has the same structure as the first vibration part 51. Specifically, the third vibration part 53 has a flat third inner surface 53a that is opposed to the first inner surface 51a with the optical fiber 2 sandwiched therebetween in an X radial direction and that is made to abut against the outer periphery of the optical fiber 2. An electrode 73a to which another GND lead 8G is connected is formed on the inner surface 53a of the third vibration part 53. An electrode 73b to which another phase-A lead 8A is connected is formed on an outer surface 53b of the third vibration part 53. Accordingly, a third piezoelectrically active region 53c that expands and contracts in the Z-direction is formed between the inner surface 53a and the outer surface 53b.

The fourth vibration part 54 has the same structure as the second vibration part 52. Specifically, the fourth vibration part 54 has a flat fourth inner surface 54a that is opposed to the second inner surface 52a with the optical fiber 2 sandwiched therebetween in a Y radial direction and that is made to abut against the outer periphery of the optical fiber 2. An electrode 74a that is connected to the electrode 73a is formed on the inner surface 54a of the fourth vibration part 54. An electrode 74b to which another phase-B lead 8B is connected is formed on an outer surface 54b of the fourth vibration part 54. Accordingly, a fourth piezoelectrically active region 54c that expands and contracts in the Z-direction is formed between the inner surface 54a and the outer surface 54b.

In the assembly process of the optical fiber scanner, the relative positions of the optical fiber 2 and the first fixed member 3 are set so as to make the outer periphery of the optical fiber 2 abut against the first and second inner surfaces 51a and 52a. Then, the second fixed member 301 is positioned with respect to the first fixed member 3 and the optical fiber 2 so as to make the third and fourth inner surfaces 53a and 54a abut against the outer periphery of the optical fiber 2. At this time, the shapes of both end sections of the second fixed member 301 in the circumferential direction are designed such that the end section of the first vibration part 51 and the end section of the fourth vibration part 54, which are adjacent in the circumferential direction, are brought into close contact with each other, and the end section of the second vibration part 52 and the end section of the third vibration part 53, which are adjacent in the circumferential direction, are brought into close contact with each other. Next, the adhesive agent is hardened, thereby fixing the fixed members 3 and 301 to the outer periphery of the optical fiber 2.

According to this modification, the optical fiber 2 can be protected by the fixed members 3 and 301, which cover the outer periphery of the optical fiber 2 over the entire circumference. The optical fiber 2 is made to vibrate through stretching vibrations of the two piezoelectrically active regions 51c and 53c in the X-direction and the two piezoelectrically active regions 52c and 54c in the Y-direction, thereby making it possible to obtain a larger vibration amplitude.

### Second Embodiment

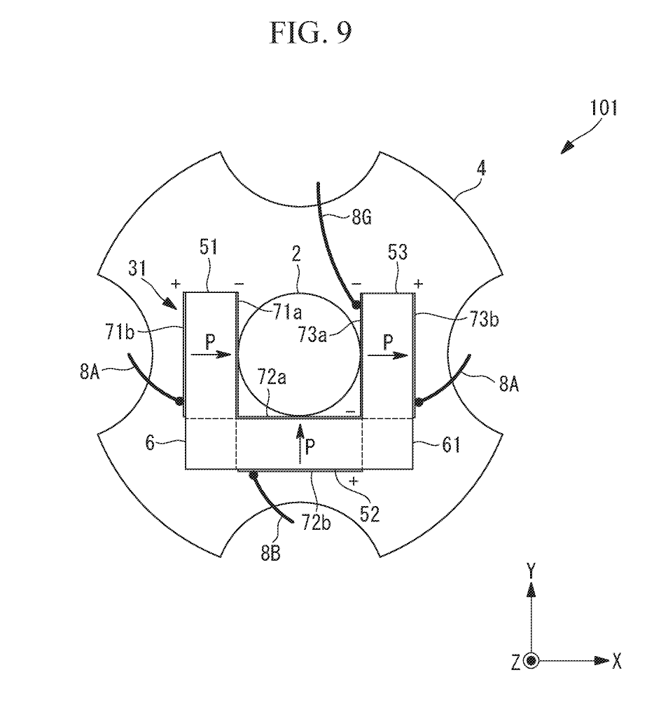

Next, an optical fiber scanner 101, an illumination device, and an observation device according to a second embodiment of the present invention will be described with reference to FIGS. 9 to 12. In this embodiment, a description will be mainly given of configurations different from those in the first embodiment, identical signs will be assigned to the configurations common to those in the first embodiment, and a description thereof will be omitted.

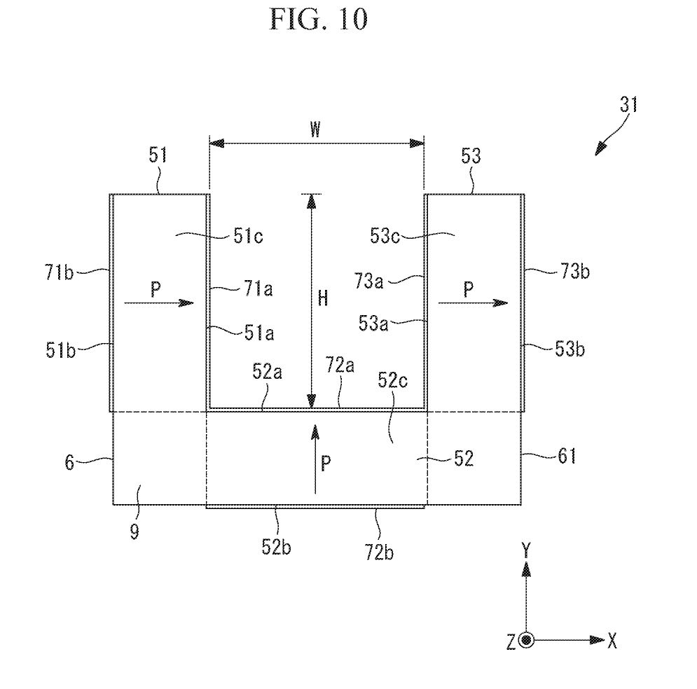

As shown in FIGS. 9 and 10, the optical fiber scanner 101 of this embodiment differs from that of the first embodiment in that a fixed member 31 further has: a third vibration part 53 that is adjacent to the optical fiber 2 in the X-direction at the opposite side from the first vibration part 51; and another connector 61 that connects the third vibration part 53 and the second vibration part 52.

The connector 61 is provided between an end section of the third vibration part 53 that is located close to the second vibration part 52 and an end section of the second vibration part 52 that is located close to the third vibration part 53. Therefore, the cross-sectional shape of the fixed member 31 in an XY-plane is a U-shape having two right-angled corners, and the fixed member 31 is open on the opposite side of the optical fiber 2 from the second vibration part 52. The fixed member 31 is made of an entirely-homogeneous piezoelectric material (for example, lead zirconate titanate) and has a seamless integral structure. The fixed member 31 is manufactured by being cut out from a prismatic piezoelectric material, for example.

The third vibration part 53 has: a flat third inner surface 53a that abuts against the outer periphery of the optical fiber 2; and a third outer surface 53b that is located closer to an outer side than the third inner surface 53a is in the X-direction and that is opposed to the third inner surface 53a. Therefore, the optical fiber 2 is disposed in a space surrounded by the three inner surfaces 51a, 52a, and 53a, and the outer periphery of the optical fiber 2 is supported by the inner surfaces 51a, 52a, and 53a at three points that are shifted by 90 degrees in the circumferential direction.

The width dimension W, in the X-direction, of the opening on the opposite side of the optical fiber 2 from the second vibration part 52 is equal to or larger than the diameter of the optical fiber 2. Therefore, in the assembly process of the optical fiber scanner 101, the optical fiber 2 can be inserted, in a radial direction, into the space surrounded by the three inner surfaces 51a, 52a, and 53a.

The third inner surface 53a is disposed parallel to the Z-direction, along a tangent to the outer periphery of the optical fiber 2 in the Y-direction. The height dimension H of each of the first inner surface 51a and the third inner surface 53a in the Y-direction is larger than the radius of the optical fiber 2 such that the central axis of the optical fiber 2 is located in the space surrounded by the three inner surfaces 51a, 52a, and 53a.

Electrodes 73a and 73b are formed on the third inner surface 53a and the third outer surface 53b, respectively, and the piezoelectric material is polarized in the X-direction in a region between the third inner surface 53a and the third outer surface 53b. Accordingly, a third piezoelectrically active region 53c that expands and contracts in the Z-direction through application of a voltage between the electrodes 73a and 73b is formed between the third inner surface 53a and the third outer surface 53b. The electrodes 73a and 73b are each disposed across the central axis of the optical fiber 2 in the Y-direction. It is preferred that the electrodes 73a and 73b each be disposed such that the sizes thereof on both sides of the central axis of the optical fiber 2 become equal. As in the electrodes 71a, 72a, 71b, and 72b, which are shown in FIG. 5, the electrodes 73a and 73b may also be formed only on partial sections of the inner surface 53a and the outer surface 53b, respectively.

Another phase-A lead 8A is connected to the electrode 73b. The drive control device 70 applies a phase-A alternating voltage to the electrode 73b via the phase-A lead 8A. Here, the polarization direction of the first piezoelectrically active region 51c and the polarization direction of the third piezoelectrically active region 53c are directed in the same side in the X-direction. Therefore, when phase-A alternating voltages are simultaneously applied to the electrode 71b and the electrode 73b, one of the first piezoelectrically active region 51c and the third piezoelectrically active region 53c contracts in the Z-direction, and the other expands in the Z-direction, thereby exciting an X-direction bending vibration at the protrusion 2a of the optical fiber 2.

The thickness dimension of each of the first and third piezoelectrically active regions 51c and 53c in the X-direction (i.e., the distance between the electrodes 71a and 71b and the distance between the electrodes 73a and 73b) and the thickness dimension of the second piezoelectrically active region 52c in the Y-direction (i.e., the distance between the electrodes 72a and 72b) are equal to each other. The drive control device 70 supplies, to the electrode 72b, a phase-B alternating voltage that is two times larger than a phase-A alternating voltage. Accordingly, the amplitude of the X-direction bending vibration due to the first vibration part 51 and the third vibration part 53 and the amplitude of the Y-direction bending vibration due to the second vibration part 52 are equal to each other, thus forming the entire light scanning trajectory into a perfectly circular shape.

In order to assemble the optical fiber scanner 101, an adhesive agent is applied to the first inner surface 51a, the second inner surface 52a, and the third inner surface 53a of the fixed member 31. Next, the optical fiber 2 is inserted, in a radial direction, into the space surrounded by the three inner surfaces 51a, 52a, and 53a, and the relative positions of the optical fiber 2 and the fixed member 31 are set so as to make the outer periphery of the optical fiber 2 abut against all of the first inner surface 51a, the second inner surface 52a, and the third inner surface 53a. Next, the adhesive agent is hardened, thus fixing the fixed member 31 to the outer periphery of the optical fiber 2. Accordingly, the optical fiber 2 and the fixed member 31 can be assembled.

In this case, according to this embodiment, the three vibration parts 51, 52, and 53, which cause the optical fiber 2 to undergo the bending vibration in the X-direction and the Y-direction, are sections of the fixed member 31, which is formed of a single member. Specifically, the relative positions of the first vibration part 51, the second vibration part 52, and the third vibration part 53 are fixed. The outer periphery of the optical fiber 2 is made to abut against the three inner surfaces 51a, 52a, and 53a of the fixed member 31, thereby positioning the optical fiber 2 at a predetermined position with respect to the fixed member 31. Therefore, the relative positions of the first vibration part 51, the second vibration part 52, the third vibration part 53, and the optical fiber 2 can be uniquely set. Accordingly, there is an advantage in that it is possible to improve the assembly accuracy of the optical fiber scanner 101 and to stably manufacture the optical fiber scanner 101 having a desired scanning performance.

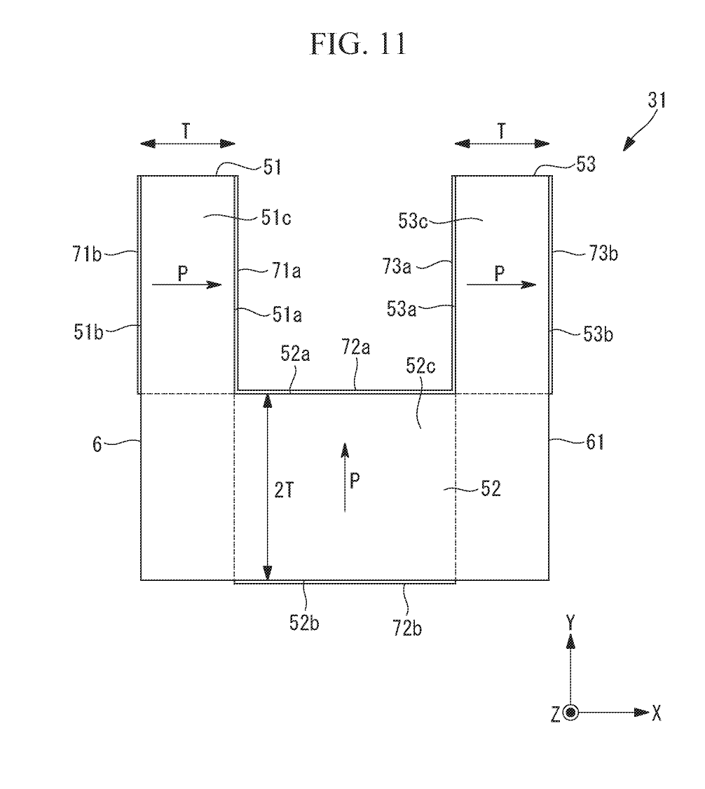

In this embodiment, although the thickness dimensions of the three piezoelectrically active regions 51c, 52c, and 53c are made equal, instead of this, as shown in FIG. 11, the second piezoelectrically active region 52c may have a larger thickness dimension than the first and third piezoelectrically active regions 51c and 53c.

By doing so, because the resonant frequency of a bending vibration of the protrusion 2a in the X-direction and the resonant frequency of a bending vibration thereof in the Y-direction become close to each other, it is possible to cause the protrusion 2a to stably undergo the bending vibrations and to obtain a more stable scanning trajectory B.

In a case in which the second piezoelectrically active region 52c has a thickness dimension that is two times larger than each of those of the first and third piezoelectrically active regions 51c and 53c, when the magnitudes of phase-A and phase-B alternating voltages are equal, the amplitudes of bending vibrations of the protrusion 2a in the X-direction and the Y-direction are equal. Specifically, alternating voltages having equal magnitudes can be supplied to all of the electrodes 71b, 72b, and 73b, thus making it easy to control the alternating voltages.

In this embodiment, a support member that is made of a material other than the piezoelectric material may be provided instead of the third vibration part 53. In this case, the support member has an inner surface (third inner surface) that is brought into contact with the outer periphery of the optical fiber 2, such that the outer periphery of the optical fiber 2 is supported by the fixed member 31 at three points in the circumferential direction.

In this embodiment, the connector 6, 61 may also be provided with the vibration absorbing member 9, such as that shown in FIG. 6 or 7.

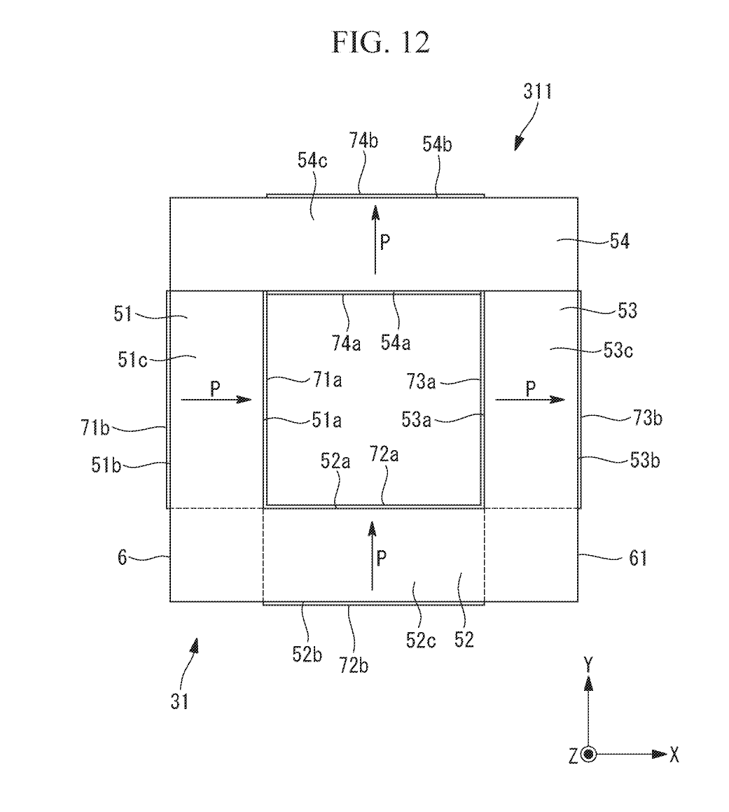

In this embodiment, although the single fixed member 31 is provided, instead of this, as shown in FIG. 12, it is also possible to further provide another fixed member (second fixed member) 311 that is disposed adjacent to the fixed member (first fixed member) 31 in the circumferential direction, so as to form a square tube for surrounding the optical fiber 2 together with the fixed member 31.

The second fixed member 311 is provided with a fourth vibration part 54 that has a flat-plate shape in which a flat inner surface 54a is brought into contact with the outer periphery of the optical fiber 2 and that is adjacent to the optical fiber 2 in the Y-direction at the opposite side from the second vibration part 52.

The fourth vibration part 54 has the same structure as the fourth vibration part 54 that is described in the first embodiment and that is shown in FIG. 8. Specifically, the fourth vibration part 54 of this modification has the flat fourth inner surface 54a that is opposed to the second inner surface 52a with the optical fiber 2 sandwiched therebetween in the Y-radial direction, and a fourth piezoelectrically active region 54c that expands and contracts in the Z-direction is formed between the inner surface 54a and an outer surface 54b.

In the assembly process of the optical fiber scanner, after the relative positions of the optical fiber 2 and the first fixed member 31 are set such that the outer periphery of the optical fiber 2 abuts against the first, second, and third inner surfaces 51a, 52a, and 53a. Then, the second fixed member 311 is positioned with respect to the first fixed member 31 and the optical fiber 2 such that the fourth inner surface 54a abuts against the outer periphery of the optical fiber 2. At this time, the shapes of both end sections of the second fixed member 311 are designed such that the end section of the first vibration part 51 and the end section of the fourth vibration part 54, which are adjacent in the circumferential direction, are brought into close contact with each other, and the end section of the third vibration part 53 and the end section of the fourth vibration part 54, which are adjacent in the circumferential direction, are brought into close contact with each other. Next, an adhesive agent is hardened, thereby fixing the fixed members 31 and 311 to the outer periphery of the optical fiber 2.

According to this modification, the optical fiber 2 can be protected by the fixed members 3 and 311, which cover the outer periphery of the optical fiber 2 over the entire circumference. The optical fiber 2 is made to vibrate through stretching vibrations of the two piezoelectrically active regions 51c and 53c in the X-direction and the two piezoelectrically active regions 52c and 54c in the Y-direction, thereby making it possible to obtain a larger vibration amplitude.

In the above-described first and second embodiments, it is also possible to use an optical fiber 2 whose outer periphery is coated with a metal coating from the base end thereof to a section thereof in the vicinity of the distal end of the fixed member 3, 31.

By doing so, a suitable solder for precise joining can be used to join the optical fiber 2 and the inner surface 51a, 52a, 53a of the fixed member 3, 31, thus making it possible to further improve the assembly accuracy of the optical fiber scanner 1, 101. The optical fiber 2 can be protected by the metal coating, thus making it possible to prevent the optical fiber 2 from being damaged during assembly. Because the electrode 71a, 72a, 73a on the inner surface 51a, 52a, 53a is electrically connected to the fixing part 4 via the metal coating, a single GND lead 8G is connected to the fixing part 4, instead of the electrode 71a, 72a, 73a, thereby making it possible to cause the fixing part 4 to function as a common GND electrode. Accordingly, there is an advantage in that the wiring operation for the lead 8G can be facilitated.

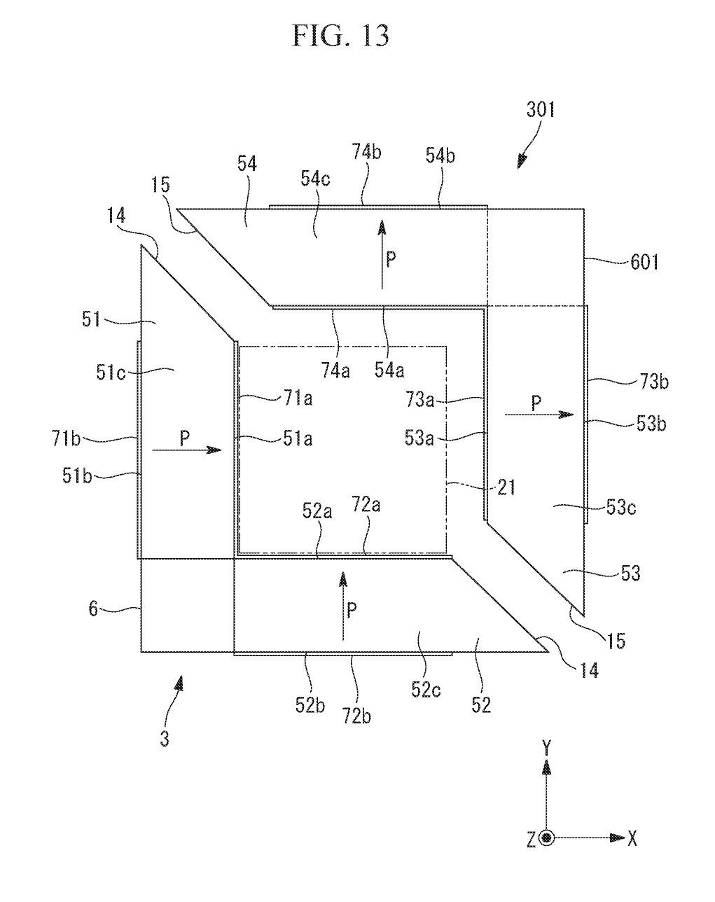

In the above-described first and second embodiments, when the two fixed members 3 and 301 shown in FIG. 8, which form a square tube, are provided or when the two fixed members 31 and 311 shown in FIG. 12, which form a square tube, are provided, it is also possible to provide chamfers 14 and 15 at the end sections of the respective fixed members 3 and 301 in the circumferential direction, as shown in FIG. 13.

The chamfers 14 and 15 are formed so as to be brought into close contact with each other when the first fixed member 3 and the second fixed member 301 are assembled into a square tube shape. FIG. 13 shows the flat chamfers 14 and 15. The chamfers 14 and 15 may each have a concavo-convex shape or a curved surface. The chamfers 14 and 15 may be formed over the entire lengths of the fixed members 3 and 301 in the longitudinal direction or may be formed only over partial lengths thereof in the longitudinal direction.

By providing the chamfers 14 and 15 in this way, the two fixed members 3 and 301 can be easily assembled so as to be in close contact with each other.

In the above-described first and second embodiments, although the optical fiber 2 is cylindrical, instead of this, as shown in FIG. 13, it is also possible to use a square-prism-shaped optical fiber 21 that has two side surfaces opposed in the X-direction and two side surfaces opposed in the Y-direction. The optical fiber 21 is positioned with respect to the fixed member such that at least two side surfaces thereof abut against the two inner surfaces 51a and 52a of the fixed member. Although FIG. 13 shows an example combination of the square-prism-shaped optical fiber 21 and the two fixed members 3 and 301, it is also possible to combine the optical fiber 21 with any of the above-described fixed members.

In this way, by using the square-prism-shaped optical fiber 21, the vibration of the optical fiber 2 in the X-direction and the vibration thereof in the Y-direction can be reliably separated from each other.

As a result, the following aspects are derived from the above-described embodiments.

According to a first aspect, the present invention provides an optical fiber scanner including: an optical fiber that has a longitudinal axis and that emits light from a distal end portion thereof; and a fixed member that is fixed to an outer periphery of the optical fiber at a position closer to a base end portion of the optical fiber than to the distal end portion thereof, wherein the fixed member is provided with: a first vibration part that is adjacent to the optical fiber in a first radial direction of the optical fiber and that expands and contracts in the longitudinal direction of the optical fiber when a voltage is applied thereto; a second vibration part that is adjacent to the optical fiber in a second radial direction of the optical fiber, the second radial direction intersecting with the first radial direction, and that expands and contracts in the longitudinal direction of the optical fiber when a voltage is applied thereto; and a connector that connects the first vibration part and the second vibration part; the first vibration part has a first inner surface that abuts against the outer periphery of the optical fiber; and the second vibration part has a second inner surface that is disposed at an angle with respect to the first inner surface and that abuts against the outer periphery of the optical fiber.

According to the first aspect of the present invention, when a voltage is applied to the first vibration part, the first vibration part is deformed in the longitudinal direction of the optical fiber, thereby causing bending deformation of the optical fiber in the first radial direction and causing the distal end of the optical fiber to be displaced in the first radial direction. Accordingly, light emitted from the distal end of the optical fiber is scanned in the first radial direction. Similarly, when a voltage is applied to the second vibration part, the second vibration part is deformed in the longitudinal direction of the optical fiber, thereby causing bending deformation of the optical fiber in the second radial direction and causing the distal end of the optical fiber to be displaced in the second radial direction. Accordingly, light emitted from the distal end of the optical fiber is scanned in the second radial direction, which intersects with the first radial direction. Therefore, voltages are simultaneously applied to the first vibration part and the second vibration part, thereby making it possible to two-dimensionally scan the light.

In this case, because the first vibration part and the second vibration part are respectively formed as sections of the fixed member, which is formed of a single member, the relative positions of the first vibration part and the second vibration part are fixed. In the assembly process of the optical fiber scanner, the outer periphery of the optical fiber is made to abut against the first inner surface and the second inner surface, which form an angle, in radial directions, thereby positioning the optical fiber at a predetermined position with respect to the fixed member. Therefore, the relative positions of the three parts, i.e., the first vibration part, the second vibration part, and the optical fiber, are uniquely set. Accordingly, the assembly accuracy of the optical fiber scanner is improved, thus making it possible to obtain stable scanning performance.

In the above-described first aspect, at least one part of the connector may have a higher vibration absorption than the vibration absorption of the first vibration part and the second vibration part.

By doing so, because vibrations are absorbed at the connector between the first vibration part and the second vibration part, it is possible to prevent stretching vibrations from being transferred between the first vibration part and the second vibration part.

In the above-described first aspect, it is preferable that an outer part of the connector that is located on the opposite side from the optical fiber have a higher vibration absorption than the vibration absorption of the first vibration part and the second vibration part.

By doing so, when the vibration absorption of the connector is enhanced by using a different material from that of the first vibration part and the second vibration part, it is possible to facilitate machining of the connector.

In the above-described first aspect, the fixed member may have a third inner surface that is provided at the opposite side of the optical fiber from the first inner surface and that is brought into contact with the outer periphery of the optical fiber.

By doing so, in the assembly process of the optical fiber scanner, the optical fiber can be inserted, from an opening in a radial direction, into the space surrounded by the first inner surface, the second inner surface, and the third inner surface. The outer periphery of the optical fiber is supported by the three inner surfaces at three points that are at different positions in the circumferential direction, thereby making it possible to stably hold the optical fiber.

The above-described first aspect may further include a third vibration part that is provided at the opposite side of the optical fiber from the first vibration part and that expands and contracts in the longitudinal direction of the optical fiber when a voltage is applied thereto, wherein the third vibration part may have the third inner surface.

By doing so, it is possible to give a stronger deforming force to the optical fiber in the first radial direction by means of the first and third vibration parts.

In the above-described first aspect, the fixed member may be open, in the second radial direction, at the opposite side of the optical fiber from the second inner surface.

In the above-described first aspect, the first inner surface and the third inner surface may have a larger size than the radius of the optical fiber in the second radial direction; and an opening of the fixed member on the opposite side of the optical fiber from the second inner surface may have a larger size than the diameter of the optical fiber in the direction perpendicular to the second radial direction.

By doing so, the optical fiber can be easily inserted from the opening. The optical fiber can be disposed such that the central axis of the optical fiber is positioned in the space surrounded by the three inner surfaces.

In the above-described first aspect, the size of the second vibration part in the second radial direction may be larger than the sizes of the first vibration part and the third vibration part in the first radial direction.

By doing so, the resonant frequency of a bending vibration of the optical fiber in the first radial direction and the resonant frequency of a bending vibration of the optical fiber in the second radial direction become close to each other. Therefore, when the optical fiber is made to undergo a bending vibration by applying alternating voltages to the three vibration parts, the bending vibrations of the optical fiber can be made more stable.

In the above-described first aspect, the fixed member may include: a first fixed member that is provided with the first vibration part, the second vibration part, and the connector; and a second fixed member that is provided with a fourth vibration part having a fourth inner surface that is provided at the opposite side of the optical fiber from the second inner surface and that is brought into contact with the outer periphery of the optical fiber; and the first fixed member and the second fixed member may be disposed adjacent to each other in the circumferential direction around the longitudinal axis and may be assembled into a tube shape surrounding the outer periphery of the optical fiber, in a close contact state.

By doing so, in the assembly process of the optical fiber scanner, after the outer periphery of the optical fiber is made to abut against the first inner surface and the second inner surface of the first fixed member and is positioned, the second fixed member can be attached to the first fixed member such that the first fixed member and the second fixed member form a tube. The outer periphery of the optical fiber is supported by the four inner surfaces at four points that are at different positions in the circumferential direction, thereby making it possible to more stably hold the optical fiber.

In the above-described first aspect, the fixed member may include: a first fixed member that is provided with the first vibration part, the second vibration part, and the connector; and a second fixed member that is provided with: a third vibration part having a third inner surface that is provided at the opposite side of the optical fiber from the first inner surface and that is brought into contact with the outer periphery of the optical fiber; a fourth vibration part having a fourth inner surface that is provided at the opposite side of the optical fiber from the second inner surface and that is brought into contact with the outer periphery of the optical fiber; and a second connector that connects the third vibration part and the fourth vibration part; and the first fixed member and the second fixed member may be adjacent to each other in the circumferential direction around the longitudinal axis and may be assembled into a tube shape surrounding the outer periphery of the optical fiber, in a close contact state.

By doing so, in the assembly process of the optical fiber scanner, after the outer periphery of the optical fiber is made to abut against the first inner surface and the second inner surface of the first fixed member and is positioned, the second fixed member can be attached to the first fixed member such that the first fixed member and the second fixed member form a tube. The outer periphery of the optical fiber is supported by the four inner surfaces at four points that are at different positions in the circumferential direction, thereby making it possible to more stably hold the optical fiber.

In the above-described first aspect, the first fixed member and the second fixed member may have, at both ends thereof in the circumferential direction, chamfers that at least partially extend along the longitudinal direction; and the first fixed member and the second fixed member may be assembled in a close contact state such that the chamfers are brought into close contact with each other.

By doing so, it is possible to easily assemble the first fixed member and the second fixed member so as to bring them into close contact with each other.

In the above-described first aspect, a metal coating for coating the outer periphery of the optical fiber from the base end of the optical fiber to a section thereof in the vicinity of the distal end of the fixed member may be provided on the outer periphery of the optical fiber.

By doing so, because a suitable solder for precise joining can be used to join the fixed member and the optical fiber, the assembly accuracy can be further improved. The optical fiber can be protected by a metal coating. Because another member that is electrically connected to the respective vibration parts via the optical fiber can be used as a ground (GND) electrode, it is not necessary to directly connect a GND lead to the fixed member, and wiring of the lead can be facilitated.

In the above-described first aspect, the optical fiber may have a square-prism shape having side surfaces in the first radial direction and the second radial direction.

By doing so, a vibration of the optical fiber in the first radial direction and a vibration thereof in the second radial direction can be reliably separated from each other.

According to a second aspect, the present invention provides an illumination device including: a light source that produces illumination light; and an optical fiber scanner according to the first aspect, in which the base end of the optical fiber is connected to the light source.

According to a third aspect, the present invention provides an observation device including: an illumination device according to the second aspect; a light detector that detects return light returning from an object when the object is irradiated with illumination light from the illumination device; and a voltage supplyer that supplies the voltage to the first vibration part and the second vibration part of the fixed member.

### REFERENCE SIGNS LIST

1, 101 optical fiber scanner
2 optical fiber
3, 31 fixed member
4 fixing part
51, 52, 53 vibration part
51a, 52a, 53a inner surface
51b, 52b, 53b outer surface
51c, 52c, 53c piezoelectrically active region
6, 61 connector
71a, 71b, 72a, 72b, 73a, 73b electrode
8A, 8B, 8G lead
9 vibration absorbing member
10 illumination device
11 focusing lens
12 frame
13 optical fiber
20 endoscope
20a insertion portion
30 control device body
40 display
50 light source
60 light detector
70 drive control device (voltage supplyer)
100 observation device
A object
B scanning trajectory

1. An optical fiber scanner comprising:
an optical fiber that has a longitudinal axis and that is configured to emit light from a distal end portion thereof; and
a fixed member that is fixed to an outer periphery of the optical fiber at a position closer to a base end portion of the optical fiber than to the distal end portion thereof,
wherein the fixed member is provided with:
a first vibration part that is adjacent to the optical fiber in a first radial direction of the optical fiber and that is configured to expand and contract in the longitudinal direction of the optical fiber when a voltage is applied thereto;
a second vibration part that is adjacent to the optical fiber in a second radial direction of the optical fiber, the second radial direction intersecting with the first radial direction, and that is configured to expand and contract in the longitudinal direction of the optical fiber when a voltage is applied thereto; and
a connector that connects the first vibration part and the second vibration part;
the first vibration part has a first inner surface that abuts against the outer periphery of the optical fiber; and
the second vibration part has a second inner surface that is disposed at an angle with respect to the first inner surface and that abuts against the outer periphery of the optical fiber.

2. An optical fiber scanner according to claim 1, wherein at least one part of the connector has a higher vibration absorption than the vibration absorption of the first vibration part and the second vibration part.

3. An optical fiber scanner according to claim 2, wherein an outer part of the connector located on the opposite side from the optical fiber has a higher vibration absorption than the vibration absorption of the first vibration part and the second vibration part.

4. An optical fiber scanner according to claim 1, wherein the fixed member has a third inner surface that is provided at the opposite side of the optical fiber from the first inner surface and that is brought into contact with the outer periphery of the optical fiber.

5. An optical fiber scanner according to claim 4, further comprising a third vibration part that is provided at the opposite side of the optical fiber from the first vibration part and that is configured to expand and contract in the longitudinal direction of the optical fiber when a voltage is applied thereto,
wherein the third vibration part has the third inner surface.

6. An optical fiber scanner according to claim 4, wherein the fixed member is open, in the second radial direction, at the opposite side of the optical fiber from the second inner surface.

7. An optical fiber scanner according to claim 6,
wherein the first inner surface and the third inner surface have a larger size than the radius of the optical fiber in the second radial direction; and
an opening of the fixed member on the opposite side of the optical fiber from the second inner surface has a larger size than the diameter of the optical fiber in the direction perpendicular to the second radial direction.

8. An optical fiber scanner according to claim 6, wherein the size of the second vibration part in the second radial direction is larger than the sizes of the first vibration part and the third vibration part in the first radial direction.

9. An optical fiber scanner according to claim 4,
wherein the fixed member includes:
a first fixed member that is provided with the first vibration part, the second vibration part, and the connector; and
a second fixed member that is provided with a fourth vibration part having a fourth inner surface provided at the opposite side of the optical fiber from the second inner surface and that is brought into contact with the outer periphery of the optical fiber; and
the first fixed member and the second fixed member are disposed adjacent to each other in the circumferential direction around the longitudinal axis and are assembled into a tube shape surrounding the outer periphery of the optical fiber, in a close contact state.

10. An optical fiber scanner according to claim 1,
wherein the fixed member includes:
a first fixed member that is provided with the first vibration part, the second vibration part, and the connector; and
a second fixed member that is provided with: a third vibration part having a third inner surface that is provided at the opposite side of the optical fiber from the first inner surface and that is brought into contact with the outer periphery of the optical fiber; a fourth vibration part having a fourth inner surface that is provided at the opposite side of the optical fiber from the second inner surface and that is brought into contact with the outer periphery of the optical fiber; and a second connector that connects the third vibration part and the fourth vibration part; and
the first fixed member and the second fixed member are adjacent to each other in the circumferential direction around the longitudinal axis and are assembled into a tube shape surrounding the outer periphery of the optical fiber, in a close contact state.

11. An optical fiber scanner according to claim 9,
wherein the first fixed member and the second fixed member have, at both ends thereof in the circumferential direction, chamfers at least partially extend along the longitudinal direction; and
the first fixed member and the second fixed member are assembled in a close contact state such that the chamfered sections are brought into close contact with each other.

12. An optical fiber scanner according to claim 1, wherein a metal coating for coating the outer periphery of the optical fiber from the base end of the optical fiber to a section thereof in the vicinity of the distal end of the fixed member is provided on the outer periphery of the optical fiber.

13. An optical fiber scanner according to claim 1, wherein the optical fiber has a square-prism shape having side surfaces in the first radial direction and the second radial direction.

14. An illumination device comprising:
a light source that is configured to produce illumination light; and
an optical fiber scanner according to claim 1, in which the base end of the optical fiber is connected to the light source.

15. An observation device comprising:
an illumination device according to claim 14;
a light detector that is configured to detect return light returning from an object when the object is irradiated with illumination light from the illumination device; and
a voltage supplyer that is configured to supply the voltage to the first vibration part and the second vibration part of the fixed member.
